(19) United States
(12) Patent Application Publication
(10) Pub. No.: US 2026/0053135 A1
(43) Pub. Date: Feb. 26, 2026

(54) ANTIBACTERIAL NANOSTRUCTURED SUBSTRATE AND METHOD OF MANUFACTURING THE SAME

(71) Applicant: Quantum NIL Corporation, Hsinchu City (TW)

(72) Inventors: Chao-Min CHENG, Hsinchu City (TW); Chung-Hsiang LIN, Hsinchu City (TW); Shu-Yun SHEU, Hsinchu City (TW); Kang-Yuan LEE, Hsinchu City (TW); Robert S. LANGER, Newton, MA (US)

(21) Appl. No.: 19/219,066

(22) Filed: May 27, 2025

Related U.S. Application Data
(60) Provisional application No. 63/686,040, filed on Aug. 22, 2024.

(30) Foreign Application Priority Data
Dec. 24, 2024 (TW) 113150388

(51) Int. Cl. A01N 25/34; A01N 25/08; B82Y 5/00

(52) U.S. Cl. CPC A01N 25/34; A01N 25/08; B82Y 5/00

(57) ABSTRACT

The antibacterial nanostructured substrate comprises a substrate plate and a nanostructure composite. The nanostructure composite is formed on the first surface of the substrate plate and includes multiple first nanostructure bodies and multiple second nanostructure bodies that are adjacently arranged in intervals. The first nanostructure bodies and the second nanostructure bodies are periodically arranged, with the center position of each first nanostructure body defined as a first reference point and the center position of each second nanostructure body defined as a second reference point. The first nanostructure bodies are arranged corresponding to the center position of the second nanostructure bodies. The first reference point has a first displacement and a second displacement relative to the second reference point. A method of manufacturing the antibacterial nanostructured substrate is provided to form nanostructure composite on the substrate plate using a microfabrication process.

### CROSS-REFERENCES TO RELATED APPLICATIONS

This application claims the benefit of U.S. Provisional Application Ser. No. 63/686,040 filed on Aug. 22, 2024, and the benefit of Taiwan Patent Application Serial No. 113150388 filed on Dec. 24, 2024. The entirety of each Application is incorporated herein by reference.

### BACKGROUND OF THE INVENTION

### Field of the Invention

The present invention relates to an antibacterial nanostructured substrate and a method of manufacturing the same, particularly to an antibacterial substrate with multiple periodic nano- and microstructures and a method of manufacturing the same.

### Descriptions of the Related Art

Gram-positive and gram-negative bacteria pose significant threats to human health and can cause various infections and diseases in humans. Among gram-positive bacteria, Staphylococcus aureus is one of the most representative species, commonly associated with skin, respiratory tract, and wound infections. In particular, methicillin-resistant Staphylococcus aureus (MRSA) can cause severe and treatment-resistant diseases such as sepsis and pneumonia. On the other hand, gram-negative bacteria, including Escherichia coli, Klebsiella pneumoniae, and Pseudomonas aeruginosa, feature multilayered cell membranes, exhibit strong antibiotic resistance, and frequently cause urinary tract infections, pulmonary infections, and gastrointestinal diseases. To address these infections, antibacterial films and materials are widely used in medical devices, packaging materials, and surface coatings. Common antibacterial technologies include silver ion coatings, copper nanoparticles, titanium dioxide (TiO₂) films, and polymers with antibacterial properties.

Nanoimprint Lithography (NIL) is a technique for fabricating high-precision nanostructures. Micro- and nanoscale patterns are produced by transferring the pattern with micro/nanostructures from a mold onto a photoresist layer on a substrate. NIL offers cost-effective, high-resolution nanostructure production with a simple and rapid process, making it widely applicable in semiconductors, optical components, and biomedical fields. The main process of NIL involves mold preparation, photoresist coating, imprinting, and curing. Various curing methods are available, such as laser interference photocuring, which precisely cures the photoresist through laser interference; electron beam curing, which achieves ultra-high resolution pattern transfer using high-energy electron beams; ultraviolet (UV) curing, which utilizes UV light to expose photosensitive photoresist materials, enabling fast processing speed and low cost; thermal curing, which cures the photoresist material by heating, suitable for thermosensitive materials; and photochemical curing, which involves curing through the interaction of specific wavelengths of light with photo-initiators. Depending on the material and application requirements, an appropriate curing method can be selected to complete the transfer of nanostructures. The availability of multiple curing techniques provides flexibility to NIL technology, allowing it to be adjusted and optimized for various applications.

Current antibacterial surface or film technologies still face several challenges in practical applications. First, the fabrication of films or coatings with high antibacterial efficiency is often complex, requiring multi-layer structures or special materials such as silver ions or copper nanoparticles, which results in high production costs. Second, conventional manufacturing methods are time-consuming and involve complex steps, making it difficult to achieve rapid production, especially in large-scale applications. Moreover, the durability and stability of these antibacterial coatings may decrease under certain harsh conditions, further affecting their practicality. By introducing NIL technology, these issues are expected to be resolved. NIL technology not only enables the production of antibacterial surfaces with precise structures in a cost-effective, fast, and efficient manner, but it also enhances the antibacterial effect of the films through nanostructure design. This technology enhances the flexibility of patterning and structural design for antibacterial materials, thereby increasing surface area and contact opportunities with bacteria, while also reducing production time and costs, providing a viable solution for mass production.

In view of the above, the present invention provides a novel antibacterial nanostructured substrate and its manufacturing method to address the deficiencies of the prior art, such as poor antibacterial efficacy, limited antibacterial spectrum, high manufacturing costs, and prolonged production time.

### SUMMARY OF THE INVENTION

The objective of the present invention is to provide an antibacterial nanostructured substrate and a method of manufacturing the same. The main technical feature of the invention lies in fabricating an antibacterial nanostructured substrate with multiple periodically arranged two-dimensional nanostructures. In the present invention, a dual-periodic nanostructure is exemplified, wherein a nanostructure composite is formed on a substrate, comprising first and second nanostructure bodies, each arranged in a periodic pattern. Through the specific arrangement of different nanostructures, the antibacterial nanostructured substrate can simultaneously exhibit antibacterial effects against multiple types of bacteria while optimizing its antibacterial effectiveness. Therefore, this invention offers a novel antibacterial nanostructured substrate and its manufacturing method.

To achieve the above-mentioned objective, the present invention discloses an antibacterial nanostructured substrate and a method of manufacturing the same. The antibacterial nanostructured substrate comprises a substrate plate and a nanostructure composite. The substrate plate includes a first surface and a second surface corresponding to the first surface. The nanostructure composite is formed on the first surface of the substrate plate and includes a plurality of first nanostructure bodies and a plurality of second nanostructure bodies arranged adjacently with intervals. The first nanostructure bodies and the second nanostructure bodies are each arranged in a periodic pattern. Each of the first nanostructure bodies has a center defined as a first reference point. Each of the second nanostructure bodies has a center defined as a second reference point. The first nanostructure bodies are positioned corresponding to the centers of the second nanostructure bodies. The first reference point has a first displacement in a first direction and a second displacement in a second direction perpendicular to the first direction, relative to the second reference point.

In one embodiment of the antibacterial nanostructured substrate of the present invention, a first spacing is defined between the first reference points of two first nanostructure bodies arranged adjacent to each other. The first displacement and the second displacement are both smaller than the first spacing.

In one embodiment of the antibacterial nanostructured substrate of the present invention, the first nanostructure bodies include a plurality of first nanopillars. The second nanostructure bodies include a plurality of second nanopillars. The first nanopillars and the second nanopillars extend from the first surface.

In one embodiment of the antibacterial nanostructured substrate of the present invention, the first nanostructure bodies and the second nanostructure bodies collectively form a square lattice array or a hexagonal lattice array.

In one embodiment of the antibacterial nanostructured substrate of the present invention, the first nanostructure bodies and the second nanostructure bodies are arranged in a dual-periodic pattern.

In one embodiment of the antibacterial nanostructured substrate of the present invention, the antibacterial nanostructured substrate has a water contact angle greater than 90°.

In another embodiment of the antibacterial nanostructured substrate of the present invention, the first nanostructure bodies and the second nanostructure bodies have a rotation angle relative to the second direction.

The present invention further provides a method of manufacturing an antibacterial nanostructured substrate which comprises the following steps: (a) preparing a substrate plate; (b) forming a nanostructure composite on a first surface of the substrate plate via a microfabrication process, the nanostructure composite including a plurality of first nanostructure bodies and a plurality of second nanostructure bodies, wherein each of the first nanostructure bodies has a center defined as a first reference point, each of the second nanostructure bodies has a center defined as a second reference point, the first nanostructure bodies are positioned corresponding to the centers of the second nanostructure bodies, and the first reference point has a first displacement in a first direction and a second displacement in a second direction perpendicular to the first direction, relative to the second reference point

In one embodiment of the method of manufacturing the antibacterial nanostructured substrate of the present invention, a first spacing is defined between the first reference points of two adjacent first nanostructure bodies. The first displacement and the second displacement are both smaller than the first spacing.

In one embodiment of the method of manufacturing the antibacterial nanostructured substrate of the present invention, the first nanostructure bodies and the second nanostructure bodies collectively form a square lattice array or a hexagonal lattice array.

In one embodiment of the method of manufacturing the antibacterial nanostructured substrate of the present invention, the first nanostructure bodies and the second nanostructure bodies are arranged in a dual-periodic pattern.

In one embodiment of the method of manufacturing the antibacterial nanostructured substrate of the present invention, the antibacterial nanostructured substrate has a water contact angle greater than 90°.

In one embodiment of the method of manufacturing the antibacterial nanostructured substrate of the present invention, the first nanostructure bodies and the second nanostructure bodies have a rotation angle relative to the second direction.

In one embodiment of the present invention, the antibacterial nanostructured substrate produced by the method inhibits the growth of Escherichia coli, Klebsiella pneumoniae, Pseudomonas aeruginosa, and Staphylococcus aureus.

To achieve the aforementioned objectives, the present invention provides a novel antibacterial nanostructured substrate and a method of manufacturing the aforementioned novel antibacterial nanostructured substrate.

The detailed technology and preferred embodiments of the present invention are described in the following paragraphs, accompanied by the appended drawings, to enable those skilled in the art to fully understand the objectives, technical methods, and embodiments of the claimed invention.

### BRIEF DESCRIPTION OF THE DRAWINGS

FIG. 1 is a flowchart illustrating the steps of a method for manufacturing an antibacterial nanostructured substrate according to one embodiment of the present invention;

FIG. 2 is a scanning electron microscope (SEM) image of the antibacterial nanostructured substrate according to one embodiment of the present invention;

FIG. 3 is scanning electron microscope (SEM) image of the antibacterial nanostructured substrate according to another embodiment of the present invention;

FIG. 4 is a schematic diagram showing the water contact angle of a blank control group of the antibacterial nanostructured substrate according to one embodiment of the present invention;

FIG. 5 is a schematic diagram showing the water contact angle of the antibacterial nanostructured substrate according to one embodiment of the present invention;

FIG. 6 is a schematic diagram showing the water contact angle of the antibacterial nanostructured substrate according to another embodiment of the present invention; and

FIG. 7 is a schematic diagram of the method for manufacturing the antibacterial nanostructured substrate according to one embodiment of the present invention.

### DETAILED DESCRIPTION OF THE PREFERRED EMBODIMENT

Reference will now be made in detail to the present embodiments of the invention, examples of which are illustrated in the accompanying drawings, and are not intended to limit the present invention, applications or particular implementations described in these embodiments. Wherever possible, the same reference numbers are used in the drawings and the description to refer to the same or like parts. It shall be appreciated that, in the following embodiments and the attached drawings, elements unrelated to the present invention are omitted from depiction; and dimensional relationships among individual elements in the attached drawings are provided only for ease of understanding, but not to limit the actual scale.

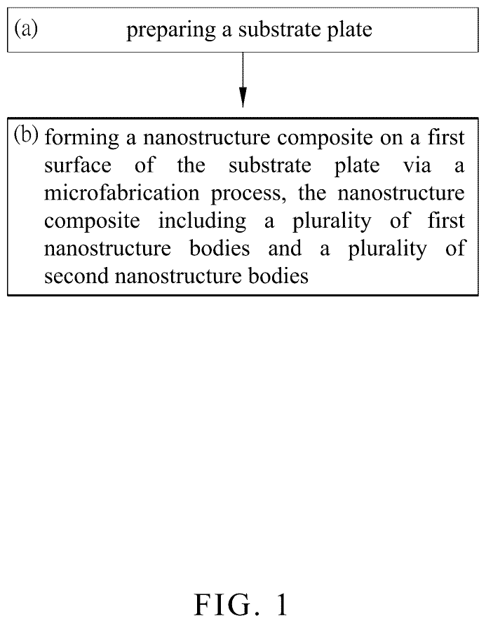

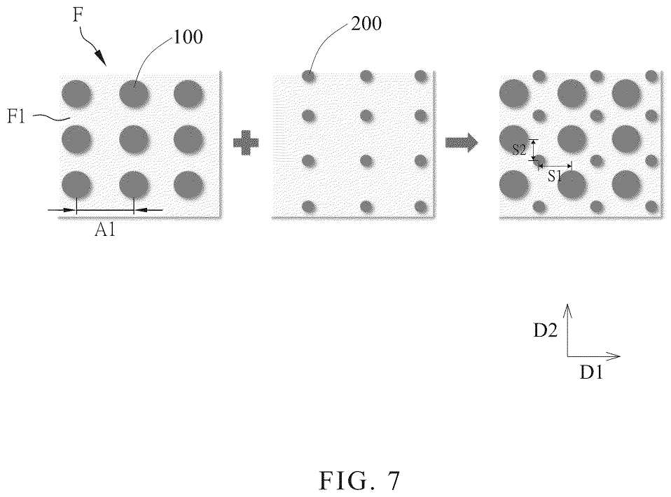

Please refer to FIG. 1 and FIG. 7, which illustrate a flowchart of the steps of a method for manufacturing an antibacterial nanostructured substrate according to the present invention. The manufacturing method comprises the following steps: (a) preparing a substrate plate F; and (b) performing a microfabrication process, which includes forming a nanostructure composite M on a first surface F1 of the substrate plate F. The nanostructure composite M comprises a plurality of first nanostructure bodies 100 and a plurality of second nanostructure bodies 200. It should be noted that, in step (b), the center of each of the first nanostructure bodies 100 is defined as a first reference point. The center of each of the second nanostructure bodies 200 is defined as a second reference point. The first nanostructure bodies 100 are arranged relative to the centers of the second nanostructure bodies 200. The first reference point has a first displacement S1 in a first direction D1 and a second displacement S2 in a second direction D2 relative to the second reference point, wherein the first direction D1 is perpendicular to the second direction D2. The following sections provide a detailed description of the steps of the manufacturing method and the experimental results.

### Example 1

### Preparation of the Antibacterial Nanostructured Substrate of the Present Invention.

First, a substrate plate F is prepared, wherein the substrate plate F has a first surface F1 and a second surface F2 corresponding to the first surface F1 (not shown in the figure). In this embodiment, the substrate plate F is exemplified as being made of plastic material, which is primarily characterized by forming a microstructured surface with nanostructures. The material of the substrate plate F is not limited thereto. Next, a microfabrication process is performed on the substrate plate F. In this embodiment, the microfabrication process involves curing a photoresist using a high-energy electron beam to pattern nanostructures, followed by the formation of a nanostructure composite M on the first surface F1 of the substrate plate F using Nanoimprint Lithography (NIL). The nanostructure composite M includes a plurality of first nanostructure bodies 100 and a plurality of second nanostructure bodies 200. In this step, the center of each first nanostructure body 100 is defined as a first reference point, and the center of each second nanostructure body 200 is defined as a second reference point. The first nanostructure bodies 100 are arranged relative to the centers of the second nanostructure bodies 200. Each of the first reference points has a first displacement S1 in a first direction D1 and a second displacement S2 in a second direction D2 relative to the second reference point, with the first direction D1 being perpendicular to the second direction D2. Finally, the first nanostructure bodies 100 and the second nanostructure bodies 200, each arranged periodically, are combined to uniformly form a microstructure on the first surface F1 of the substrate plate F.

### Example 2

Characteristics of the antibacterial nanostructured substrate.

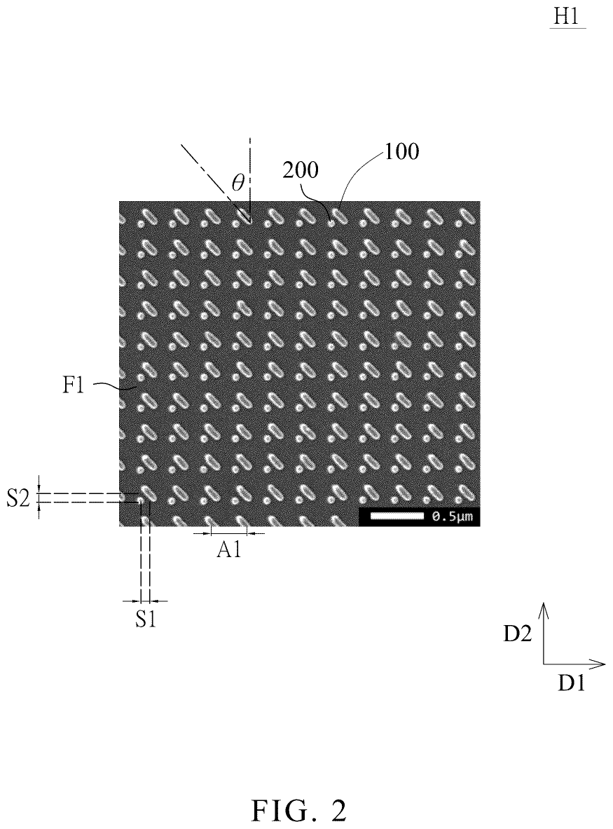

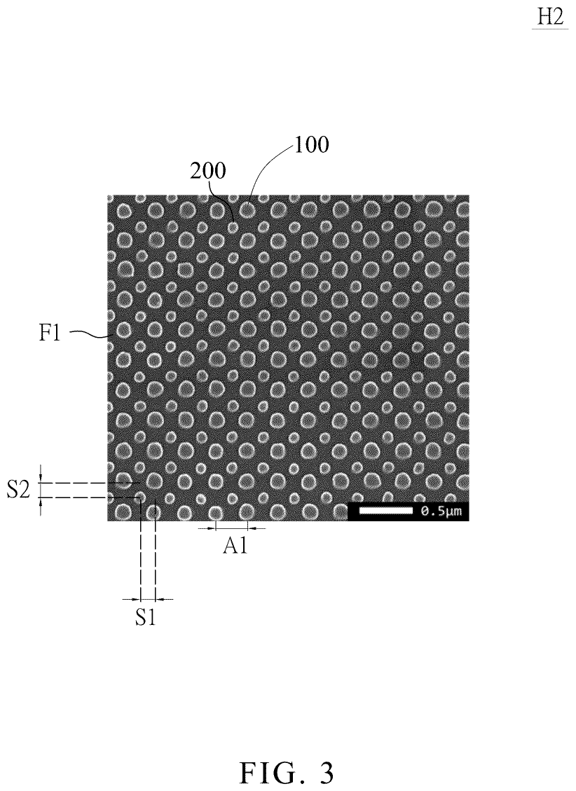

The fabricated antibacterial nanostructured substrate can be examined using a scanning electron microscope (SEM) to observe its nanostructure. Please refer to FIG. 2 and FIG. 3, which respectively illustrate SEM images of antibacterial nanostructured substrates in different embodiments of the present invention. As shown in the figures, the antibacterial nanostructured substrate primarily comprises nanostructures with dual periodicity, in other words, comprising two types of nanostructures arranged with identical periodicity. It should be noted that, in the embodiments of the present invention, the nanostructures are exemplified with identical periodicity, which can be adjusted to different periodicity based on actual requirements and are not limited thereto. As illustrated in FIG. 2 and FIG. 3, the nanostructure composite M of the antibacterial nanostructured substrate further includes a plurality of first nanostructure bodies 100 and a plurality of second nanostructure bodies 200, which differ in sizes. Specifically, the first nanostructure bodies 100 comprise a plurality of first nanopillars, and the second nanostructure bodies 200 comprise a plurality of second nanopillars. The first nanopillars and the second nanopillars extend to form on the first surface F1 (as shown in FIG. 3). It is worth noting that the first nanostructure bodies 100 and the second nanostructure bodies 200 may also be configured in the shape of nanorods (as shown in FIG. 2). The larger-sized structures correspond to the first nanostructure bodies 100, while the smaller-sized structures correspond to the second nanostructure bodies 200, which are uniformly arranged on the first surface F1. The first nanostructure bodies 100 and the second nanostructure bodies 200 share an identical periodicity (with a period of 282 nm). Further, the center of each of the first nanostructure bodies 100 is defined as a first reference point. Each of the first nanostructure bodies 100 is positioned corresponding to the center of a corresponding second nanostructure bodies 200. The center of each of the second nanostructure bodies 200 is defined as a second reference point. The first reference point has a first displacement S1 relative to the second reference point in a first direction D1, and a second displacement S2 relative to the second reference point in a second direction D2, wherein the first direction D1 is perpendicular to the second direction D2. Furthermore, a first spacing A1 is defined between the first reference points of two adjacent first nanostructure bodies 100. For example, this first spacing A1 corresponds to the period of the first nanostructure bodies 100. In this embodiment, both the first displacement S1 and the second displacement S2 are smaller than the first spacing A1. In other words, the distance between the second nanostructure 200 and the adjacent first nanostructure 100 is smaller than the periodic spacing of the first nanostructure bodies 100. As shown in FIG. 3, in the antibacterial nanostructured substrate H2 of the present invention, both the first displacement S1 and the second displacement S2 are half of the first spacing A1. This indicates that the first nanostructure bodies 100 are displaced relative to the second nanostructure bodies 200 by half of the first spacing A1 in the first direction D1 and the second direction D2, thereby forming the antibacterial nanostructured substrate with a dual-periodic nanostructure combination. Similarly, as shown in FIG. 2, in the antibacterial nanostructured substrate H1 of the present invention, both the first displacement S1 and the second displacement S2 are smaller than the first spacing A1 and less than half of the first spacing A1. In this case, the first nanostructure body 100 is positioned closer to the second nanostructure body 200 (with an approximate spacing of 60 nm). Referring again to FIG. 2 and FIG. 3, in both embodiments of the present invention, the first nanostructure bodies 100 and the second nanostructure bodies 200 are exemplarily arranged in a square lattice array. This arrangement can be modified to a hexagonal lattice array (not shown in the figures) based on actual requirements and is not limited thereto.

It should be noted that, as shown in FIG. 2, in the structure of the antibacterial nanostructured substrate H1 of the present invention, the first nanostructure bodies 100 and the second nanostructure bodies 200 have a rotation angle θ relative to the second direction D2. In this embodiment, the rotation angle θ is exemplified as 45°, which achieves an optimal antibacterial effect. The rotation angle θ can be adjusted based on actual requirements, and is not limited thereto.

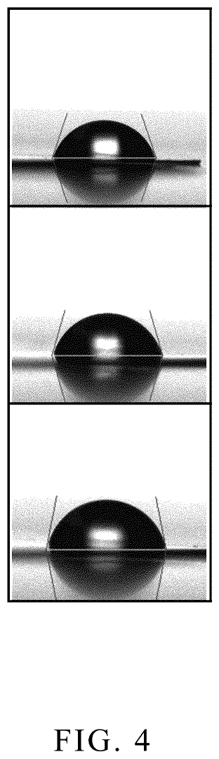

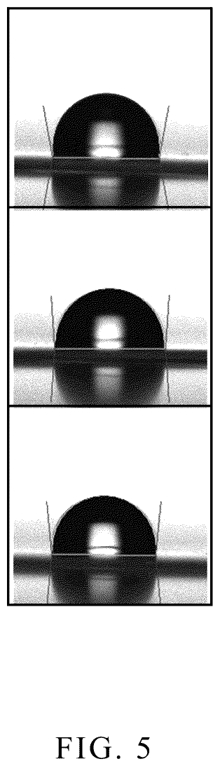

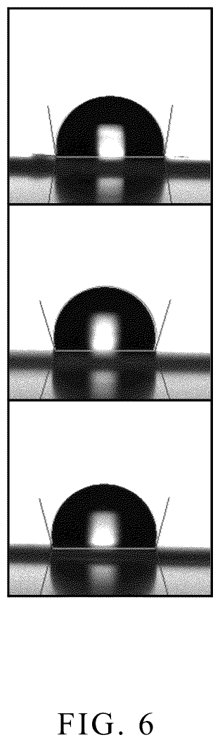

On the other hand, a water contact angle measurement experiment is conducted on the antibacterial nanostructured substrate of the present invention to confirm its hydrophilic or hydrophobic properties. Please refer to FIG. 4 to FIG. 6, which respectively illustrate schematic diagrams of the water contact angles for the blank control group, antibacterial nanostructured substrate H1, and antibacterial nanostructured substrate H2 of the present invention. After repeated measurements and averaging the water contact angles, the results indicate that the blank control group, which consists of a plain substrate without nanostructures, exhibits an average water contact angle of 74.1°. Since the water contact angle is less than 90°, the blank control group exhibits hydrophilicity. In contrast, the water contact angles of antibacterial nanostructured substrate H1 and antibacterial nanostructured substrate H2, calculated as 96.7° and 102.8° respectively, reveal that substrates incorporating nanostructures exhibit water contact angles greater than 90°, indicating a transition in material properties to hydrophobicity. Antibacterial efficacy test are subsequently conducted on the antibacterial nanostructured substrate of the present invention.

### Example 3

Antibacterial efficacy was confirmed through bacterial culture.

Antibacterial efficacy testing is conducted according to the antibacterial test method for nanostructured antibacterial products. Initially, a blank control group without nanostructures, antibacterial nanostructured substrate H1, and antibacterial nanostructured substrate H2 are prepared in advance. These three types of substrate materials are cut into test samples of predetermined sizes, and corresponding cover films are separately prepared and cut. All materials are then immersed in 75% alcohol for sterilization. Next, freshly cultured bacteria are prepared, including gram-negative bacilli such as Escherichia coli, Klebsiella pneumoniae, and Pseudomonas aeruginosa, as well as gram-positive cocci with higher toxicity, such as Staphylococcus aureus (S. aureus), for antibacterial testing. The freshly cultured bacteria are diluted to a concentration of 2.5×10⁵ to 1.0×10⁶ CFU/mL, with quantification assisted by an ultra-micro spectrophotometer. This experiment is performed in a culture dish, with the following sequence: the antibacterial nanostructured substrate of the present invention or the blank control group is placed in the culture dish, followed by the addition of the diluted bacterial suspension, and finally covered with the cover film for incubation over 18-24 hours. Subsequently, the bacterial colonies are rinsed and re-cultured. From the rinsed colonies, 0.5 mL is taken for quantification with the ultra-micro spectrophotometer, and 10 μL is separately spread onto a culture dish and incubated for 24 hours for observation. After a period of incubation, the cultured dish is processed using ImageJ (image processing software) to analyze images and calculate the ratio of the bacterial growth area to the total area of the culture medium, thereby quantifying the extent of bacterial growth. After the growth area is calculated, the values obtained from the blank control group are subtracted for comparative analysis, with the results presented in Table 1 below.

TABLE 1

Antibacterial Efficacy

| | Antibacterial | Antibacterial |
|---|---|---|
| | nanostructured | nanostructured |
| | substrate H1 | substrate H2 |
| E. coli | Reduced by 25% | Reduced by 11% |
| K. pneumoniae | Reduced by 15% | Reduced by 27% |
| P. aeruginosa | Reduced by 7% | Reduced by 9% |
| S. aureus | Reduced by 9% | Reduced by 2% |

The results in Table 1 show that both Antibacterial nanostructured substrate H1 and Antibacterial nanostructured substrate H2 exhibit antibacterial efficacy compared to the blank control group. Antibacterial nanostructured substrate H1 reduces colony counts of gram-negative bacilli by 25%, 15%, and 7% for Escherichia coli (E. coli), Klebsiella pneumoniae (K. pneumoniae), and Pseudomonas aeruginosa (P. aeruginosa), respectively. On the other hand, Antibacterial nanostructured substrate H2 reduces colony counts of E. coli, K. pneumoniae, and P. aeruginosa by 11%, 27%, and 9%, respectively. Additionally, for the gram-positive coccus Staphylococcus aureus (S. aureus), which is more virulent and less frequently studied, both Antibacterial nanostructured substrate H1 and Antibacterial nanostructured substrate H2 exhibit notable antibacterial efficacy, reducing colony counts by 9% and 2%, respectively. These results demonstrate that the novel antibacterial nanostructured substrate of the present invention indeed achieves significant antibacterial efficacy, particularly against gram-positive cocci.

In summary, the present invention provides an antibacterial nanostructured substrate and its manufacturing method. The antibacterial nanostructured substrate is primarily characterized by a nanostructure composite with periodically arranged nanostructures. In other words, it is a nanostructure composite comprising multiple periodically arranged nanostructures. The spacing, size, and arrangement direction of the multiple nanostructures in this antibacterial nanostructured substrate can be adjusted based on actual antibacterial efficacy to achieve optimized antibacterial performance. Experimental results show that the antibacterial nanostructured substrate of the present invention effectively inhibits bacterial growth against both various gram-negative bacteria and gram-positive bacterium such as Staphylococcus aureus. This nanostructured substrate can be further applied in various fields such as food, biomedicine, and biomedical detection.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims.

What is claimed is:

1. A antibacterial nanostructured substrate, comprising:
a substrate plate including a first surface and a second surface corresponding to the first surface; and
a nanostructure composite formed on the first surface of the substrate plate, including a plurality of first nanostructure bodies and a plurality of second nanostructure bodies arranged adjacently with intervals;
wherein the first nanostructure bodies and the second nanostructure bodies are each arranged in a periodic pattern, each of the first nanostructure bodies having a center defined as a first reference point, each of the second nanostructure bodies having a center defined as a second reference point, the first nanostructure bodies are positioned corresponding to the centers of the second nanostructure bodies, and the first reference point has a first displacement in a first direction and a second displacement in a second direction perpendicular to the first direction relative to the second reference point.

2. The antibacterial nanostructured substrate of claim 1, wherein a first spacing is defined between the first reference points of two first nanostructure bodies arranged adjacent to each other, and the first displacement and the second displacement are both smaller than the first spacing.

3. The antibacterial nanostructured substrate of claim 1, wherein the first nanostructure bodies include a plurality of first nanopillars, the second nanostructure bodies include a plurality of second nanopillars, and the first nanopillars and the second nanopillars extend from the first surface.

4. The antibacterial nanostructured substrate of claim 1, wherein the first nanostructure bodies and the second nanostructure bodies collectively form a square lattice array or a hexagonal lattice array.

5. The antibacterial nanostructured substrate of claim 1, wherein the first nanostructure bodies and the second nanostructure bodies are arranged in a dual-periodic pattern.

6. The antibacterial nanostructured substrate of claim 1, wherein the antibacterial nanostructured substrate has a water contact angle greater than 90°.

7. The antibacterial nanostructured substrate of claim 1, wherein the first nanostructure bodies and the second nanostructure bodies have a rotation angle relative to the second direction.

8. A method of manufacturing an antibacterial nanostructured substrate, comprising:
(a) preparing a substrate plate; and
(b) forming a nanostructure composite on a first surface of the substrate plate via a microfabrication process, the nanostructure composite including a plurality of first nanostructure bodies and a plurality of second nanostructure bodies, wherein each of the first nanostructure bodies has a center defined as a first reference point, each of the second nanostructure bodies has a center defined as a second reference point, the first nanostructure bodies are positioned corresponding to the centers of the second nanostructure bodies, and the first reference point has a first displacement in a first direction and a second displacement in a second direction perpendicular to the first direction relative to the second reference point.

9. The method of claim 8, wherein a first spacing is defined between the first reference points of two adjacent first nanostructure bodies, and both the first displacement and the second displacement are smaller than the first spacing.

10. The method of claim 8, wherein the first nanostructure bodies and the second nanostructure bodies collectively form a square lattice array or a hexagonal lattice array.

11. The method of claim 8, wherein the first nanostructure bodies and the second nanostructure bodies are arranged in a dual-periodic pattern.

12. The method of claim 8, wherein the antibacterial nanostructured substrate has a water contact angle greater than 90°.

13. The method of claim 8, wherein the first nanostructure bodies and the second nanostructure bodies have a rotation angle relative to the second direction.

14. The method of claim 8, wherein the antibacterial nanostructured substrate inhibits the growth of Escherichia coli, Klebsiella pneumoniae, Pseudomonas aeruginosa, and Staphylococcus aureus.
